(19) United States
(12) Patent Application Publication
(10) Pub. No.: US 2026/0328922 A1
(43) Pub. Date: Oct. 22, 2026

(54) SYSTEM AND METHOD FOR NON-CONTACT PEOPLE LOCALIZATION VIA FREQUENCY-MODULATED CONTINUOUS WAVE RADAR

(71) Applicant: Yeda Research and Development Co. Ltd., Rehovot (IL)

(72) Inventors: Yonina ELDAR, Rehovot (IL); Yonathan EDER, Rehovot (IL); Yhonatan KVICH, Rehovot (IL)

(21) Appl. No.: 19/463,250

(22) Filed: Jan. 29, 2026

Related U.S. Application Data
(60) Provisional application No. 63/753,013, filed on Feb. 3, 2025.

(51) Int. Cl. G01S 13/42; A61B 5/00; A61B 5/0205; A61B 5/024; A61B 5/08

(52) U.S. Cl. CPC G01S 13/42; A61B 5/0205; A61B 5/7267; A61B 5/024; A61B 5/0816

(57) ABSTRACT

A method and system are provided for localization of subject(s). Raw measured data is provided, which corresponds to data being received from a single-channel front end of each of K receivers (K>1) of SIMO FMCW radar and is indicative of L measured data frames, each comprising N consecutive beat signals. This raw measured data is used to create, for each l-th frame, data matrix Yₗ∈ [symbol] satisfying a bilinear model; and preprocessing is performed for each l-th data matrix by: iterative estimation of the localization of the one or more subjects by exploiting joint sparsity and cardiopulmonary properties of said data matrix; and/or utilizing a trained neural network to detect and localize the subject(s) with angular resolution exceeding an angular resolution defined by the K receivers. Then, support recovery data is generated being indicative of range and angle indices of the one or more subjects.

### TECHNOLOGICAL FIELD AND BACKGROUND

The present invention is in the general field of non-contact people localization via radar.

The rising demand for multi-person non-contact vital signs monitoring (NCVSM) in healthcare highlights the potential of radar technology, especially in cluttered environments. Single-input multiple-output (SIMO) frequency modulated continuous-wave (FMCW) radars enable multi-object localization, which is crucial for multi-person NCVSM. However, detecting and positioning humans in crowded scenarios is challenging due to resolution limitations.

### BACKGROUND ART

References considered to be relevant as background to the presently disclosed subject
1. F. Fioranelli, J. Le Kernec, and S. A. Shah, “Radar for health care: Recognizing human activities and monitoring vital signs,” IEEE Potentials, vol. 38, no. 4, pp. 16-23, 2019.
2. A. Ahmad, J. C. Roh, D. Wang, and A. Dubey, “Vital signs monitoring of multiple people using a fmcw millimeter-wave sensor,” in 2018 IEEE Radar Conference (RadarConf18). IEEE, 2018, pp. 1450-1455.
3. K. Han and S. Hong, “Detection and localization of multiple humans based on curve length of i/q signal trajectory using mimo fmcw radar,” IEEE Microwave and Wireless Components Letters, vol. 31, no. 4, pp. 413-416, 2021.
4. Y. Eder, Z. Liu, and Y. C. Eldar, “Sparse non-contact multiple people localization and vital signs monitoring via fmew radar,” in ICASSP 20232023 IEEE International Conference on Acoustics, Speech and Signal Processing (ICASSP). IEEE, 2023, pp. 1-5.
5. Y. Eder and Y. C. Eldar, “Sparsity-based multi-person non-contact vital signs monitoring via fmcw radar,” IEEE journal of biomedical and health informatics, vol. 27, no. 6, pp. 2806-2817, 2023.
6. X. Gao, G. Xing, S. Roy, and H. Liu, “Experiments with mm-wave automotive radar test-bed,” in 2019 53rd Asilomar conference on signals, systems, and computers. IEEE, 2019, pp. 1-6.
7. K. Konno, M. Nango, N. Honma, K. Nishimori, N. Takemura, and T. Mitsui, “Experimental evaluation of estimating living-body direction using array antenna for multipath environment,” IEEE Antennas and Wireless Propagation Letters, vol. 13, pp. 718-721, 2014.
8. B.-s. Kim, Y. Jin, J. Lee, and S. Kim, “Low-complexity music-based direction-of-arrival detection algorithm for frequency-modulated continuous-wave vital radar,” Sensors, vol. 20, no. 15, p. 4295, 2020.
9. U. Rossman, R. Tenne, O. Solomon, I. Kaplan-Ashiri, T. Dadosh, Y. C. Eldar, and D. Oron, “Rapid quantum image scanning microscopy by joint sparse reconstruction,” Optica, vol. 6, no. 10, pp. 1290-1296, 2019.
10. S. Schellenberger, K. Shi, T. Steigleder, A. Malessa, F. Michler, L. Hameyer, N. Neumann, F. Lurz, R. Weigel, C. Ostgathe et al., “A dataset of clinically recorded radar vital signs with synchronised reference sensor signals,” Scientific data, vol. 7, no. 1, p. 291, 2020.
11. C. Wang, D. Zhu, L. Sun, C. Han, and J. Guo, “Real-time through-wall multi-human localization and behavior recognition based on mimo radar,” IEEE Transactions on Geoscience and Remote Sensing, vol. 61, pp. 1-12, 2023.
12. M. Alizadeh, G. Shaker, J. C. M. De Almeida, P. P. Morita, and S. Safavi Naeini, “Remote monitoring of human vital signs using mm-wave fmcw radar,” IEEE Access, vol. 7, pp. 54958-54968, 2019.
13. E. Turppa, J. M. Kortelainen, O. Antropov, and T. Kiuru, “Vital sign monitoring using fmcw radar in various sleeping scenarios,” Sensors, vol. 20, no. 22, p. 6505, 2020.
14. A. Beck and M. Teboulle, “A fast iterative shrinkage-thresholding algorithm for linear inverse problems,” SIAM journal on imaging sciences, vol. 2, no. 1, pp. 183-202, 2009.
15. D. P. Palomar and Y. C. Eldar, Convex optimization in signal processing and communications. Cambridge university press, 2010.
16. N. Shlezinger, J. Whang, Y. C. Eldar, and A. G. Dimakis, “Model-based deep learning,” Proceedings of the IEEE, vol. 111, no. 5, pp. 465-499, 2023.
17. J. Xiang, Y. Dong, and Y. Yang, “Fista-net: Learning a fast iterative shrinkage thresholding network for inverse problems in imaging,” IEEE Transactions on Medical Imaging, vol. 40, no. 5, pp. 1329-1339, 2021.
18. Y. Wang, Z. Jiang, Y. Li, J.-N. Hwang, G. Xing, and H. Liu, “Rodnet: A real-time radar object detection network cross-supervised by camera radar fused object 3d localization,” IEEE Journal of Selected Topics in Signal Processing, vol. 15, no. 4, pp. 954-967, 2021.
19. Z.-s. Han, C.-p. Wang, and Q. Fu, “Arbitrary-oriented target detection in large scene sar images,” Defence Technology, vol. 16, no. 4, pp. 933946, 2020.

Acknowledgement of the above references herein is not to be inferred as meaning that these are in any way relevant to the patentability of the presently disclosed subject matter.

### GENERAL DESCRIPTION

The present disclosure provides a novel iterative technique for subject's localization exploiting joint sparsity and cardiopulmonary properties (e.g., breathing and heartbeat patterns). Further, the technique of the present disclosure is unfolded into a neural network that preserves the data's unique features to further enhance accuracy and reduce computational cost.

Simulations containing real-world data show the network's superior performance in detecting and positioning multiple adjacent humans, outperforming existing techniques via key metrics. The approach of the present disclosure can be integrated into advanced non-contact vital signs monitoring (NCVSM) systems where accuracy and computational efficiency are paramount.

The importance of NCVSM in healthcare has grown due to a number of aspects, including the prevalence of chronic diseases, potential hazards of disease transmission, and the substantial workload on medical personnel. Radar technology is advantageous in these circumstances since it eliminates the need for individuals to wear, carry, or engage with any supplementary devices [1]. Particularly in cluttered medical environments such as clinics and hospitals, where simultaneous monitoring of multiple individuals can be beneficial, single/multiple-input multiple-output (SIMO/MIMO) frequency modulated continuous-wave (FMCW) radars possess properties that enable the spatial localization of multiple objects, such as in a range-azimuth plane [2]-[4]. Consequently, human targets can be identified for the purpose of accurately monitoring their vital signs, such as heart rate (HR) and respiratory rate (RR) [4], [5].

The detection and positioning of individuals can be particularly challenging in noisy scenarios with multiple objects (subjects/humans) in close proximity, especially when the relative distance is smaller than the theoretical resolution, such as in radar sensors with limited bandwidth or small antenna arrays. FIGS. 2A to 2K depict the Discrete Fourier Transform (DFT) spectrum of beamformed thoracic motion of human #1 with a neighboring human #2, whose angular position differs only by 15 degrees at a theoretical resolution of 30 degrees. The beamforming was carried out using angles of 0, 10, and 20 degrees relative to human #1 position. Although all angle deviations fall below the resolution limit, as the deviation increases, the vital information becomes distorted due to deflection noise and mutual interference, highlighting the importance of precise human localization as a prerequisite for accurate NCVSM.

Several studies employed the common angle-FFT technique to spatially localize objects with a mmWave FMCW radar [2], [3], [6]. This approach translates radar data into range-azimuth planes by performing 1-D FFTs over the ADC samples axis, followed by 1-D FFTs over the receiver elements axis. However, this approach is heavily influenced by reflections from static objects in the radar's field of view (FOV) and exhibits relatively poor angular resolution due to FFT processing limitations [6]. Researchers utilized the multiple signal classification (MUSIC) algorithm to estimate the direction of arrival (DOA) of individuals in an indoor setting [7], [8]. Although the method is well-known for its angular super-resolution, it requires determining the number of sources to be located, which is not always available, and it is highly sensitive to oscillating objects, which may result in erroneous estimates, especially in cluttered environments.

In the present disclosure, a model for the localization and NCVSM of multiple individuals using SIMO FMCW radar [4] is provided. Based on this model, the inventors developed an iterative algorithm for human localization that exploits joint sparsity [9] in the data, and prior knowledge of cardiopulmonary properties. The approach of the present disclosure exhibits impressive detection and positioning capabilities, yet it necessitates a substantial number of iterations. Therefore, the inventors utilize the concept of deep unfolding, which is a machine learning technique that combines iterative optimization algorithms with deep neural network. This enables to transform an iterative algorithm into a neural network with a fixed, limited number of layers. By modifying the network to exploit model-based characteristics, the model order is reduced and the accuracy is enhanced compared to its iterative counterpart and other techniques, even in a challenging scenario with closely spaced humans, below theoretical resolution limits.

Thus, according to a broad aspect of the present disclosure, it provides a radar-based localization system for use in localization of one or more subjects, the radar-based localization system being configured for signal communication with a single-input multiple-output (SIMO) frequency modulated continuous wave (FMCW) radar and comprising a processing and memory circuitry configured and operable to process raw measured data which is received from a single-channel front end of K receivers (K>1) of said SIMO FMCW radar and which is indicative of L measured data frames, each comprising N consecutive beat signals, said processing and memory circuitry comprising:
a preprocessing module configured and operable to utilize said raw measured data to create, for each l-th frame, a data matrix Yᵢ∈ [symbol] satisfying a bilinear model; and
a localization module configured and operable to process each said l-th data matrix Yₗ∈ [symbol] by carrying out at least one of the following:
   (i) iterative estimation of the localization of the one or more subjects by exploiting joint sparsity and cardiopulmonary properties of said data matrix, and
   (ii) utilizing a trained neural network to detect and localize the one or more subjects with angular resolution exceeding an angular resolution defined by the number K of receivers, and provide support recovery data indicative of range and angle indices of the one or more subjects.

The preprocessing module is configured and operable to utilize data indicative of predefined frequency and angle grids and assemble a range-associated matrix A and an angle-associated matrix B, satisfying a bilinear model: Yₗ=AXₗB+Nₗ, l=1, . . . , L, where Xₗ− [symbol] is an unknown matrix of complex amplitudes associated with reflections from various range-angle pairs, M being a number of general radial distances, P being a number of general azimuth angles with respect to the radar, and Nₗ is a matrix defined by noise.

The processing and memory circuitry may further comprise a filtering utility configured and operable to utilize prior knowledge of human typical pulse and breathing frequencies to define a filter applied on an input set of said data matrices,

$${\{ {Y}_{l} \}}_{l = 1}^{L} ,$$

to reduce non-human clutter.

In some embodiments, the iterative estimation of the localization of the one or more subjects by exploiting joint sparsity and cardiopulmonary properties of said data matrix utilizes a fast iterative soft-thresholding (FISTA) algorithm to perform the following minimization:

$$\hat{𝕏} = {arg min}_{𝕏 ∈ {ℂ}^{M \times P \times L}} \frac{1}{2 L} {\sum }_{l = 1}^{L} {‖ {Y}_{l} - A {X}_{l} B ‖}_{F}^{2} + γ {‖ 𝕏 ‖}_{2 , 1}$$

where ∥ [symbol] ∥_{2,1} refers to the sum of all l₂ norms over the frame dimension of a 3D tensor [symbol] ∈ [symbol] which concatenates the frames of

$${\{ {X}_{l} \}}_{l = 1}^{L}$$

and γ≥0 is a regularization parameter, said minimization providing the unknown set

$${\{ {X}_{l} \}}_{l = 1}^{L} .$$

In some embodiments, the iterative estimation of the localization is accelerated by using an update step where two previous estimates are linearly combined replacing

$${X}_{l}^{( i + 1 )} by {Z}_{l}^{( i + 1 )} = {X}_{l}^{( i )} + \frac{{t}^{( i )} - 1}{{t}^{( i + 1 )}} ( {X}_{l}^{( i )} - {X}_{l}^{( i - 1 )} )$$

where t⁽ⁱ⁺¹⁾=0.5 (1+\sqrt{1+4t⁽ⁱ⁾²}) initialized to t=0.

In some embodiments, the support recovery data indicative of range and angle indices of the one or more subjects is estimated by determining an average power across the frames of

$${\{ {X}_{l} \}}_{l = 1}^{L}$$

resulting in a 2D range-angle localization map.

In some embodiments, the processing of each said l-th data matrix Yₗ∈ [symbol] by utilizing the trained neural network with the predetermined number of layers comprises replacing the matrices A and B with weight matrices {Wᵢ} of appropriate sizes, said weight matrices being learned from train data.

In some embodiments, the weight matrices {Wᵢ} of appropriate sizes comprise a set

$${\{ {W}_{1}^{( i )} , {W}_{2}^{( i )} , {W}_{3}^{( i )} , {W}_{4}^{( i )} \}}_{i = 1}^{I} ,$$

I being the number of layers, wherein said weight matrices {Wᵢ} are initialized to:

$${W}_{1}^{( i )} = \frac{1}{{L}_{f}} {A}^{H} , {W}_{2}^{( i )} = {B}^{H} , {W}_{3}^{( i )} = - \frac{1}{{L}_{f}} {A}^{H} A , and {W}_{4}^{( i )} = {BB}^{H} ,$$

where L_{f} is a constant set to L_{f}=λₘₐₓ(A^{H}A)λₘₐₓ(B^{H}B) with λₘₐₓ denoting the largest singular value.

For example, the said matrix

$${W}_{3}^{( i )}$$

is omitted and said matrix

$${W}_{4}^{( i )}$$

is reinitialized to

$${\tilde{W}}_{4}^{( i )} = - \frac{N}{{L}_{f}} {BB}^{H} .$$

In some embodiments, the processing and memory circuitry comprises a filtering utility configured and operable to apply a filter on an input set of said data matrices

$${\{ {Y}_{l} \}}_{l = 1}^{L}$$

to reduce non-human clutter, said filter being learned from said training data and initialized utilizing prior knowledge on cardiopulmonary frequencies at rest.

According to another broad aspect of the present disclosure, it provides a computer-implemented method for localization of one or more subjects, the method comprising:
obtaining raw measured data corresponding to data being received from a single-channel front end of each of K receivers (K>1) of a SIMO FMCW radar and being indicative of L measured data frames, each comprising N consecutive beat signals;
utilizing said raw measured data to create, for each l-th frame, a data matrix Yₗ∈ [symbol] satisfying a bilinear model; and
performing preprocessing of each said l-th data matrix Yₗ∈ [symbol] by carrying out at least one of the following:
   (i) iterative estimation of the localization of the one or more subjects by exploiting joint sparsity and cardiopulmonary properties of said data matrix, and
   (ii) utilizing a trained neural network to detect and localize the one or more subjects with angular resolution exceeding an angular resolution defined by the K receivers; and
generating support recovery data indicative of range and angle indices of the one or more subjects.

The technique of the present disclosure was validated by simulations incorporating real-world data from static monitored individuals [10] via known detection and positioning metrics. The technique of the present disclosure is suitable for integration into advanced multi-person NCVSM systems that demand high accuracy, as well as in algorithms for people counting or behavior analysis [11].

The technique of the present disclosure advantageously provides superior detection performance. More specifically, it outperforms existing techniques in detecting and positioning multiple adjacent humans. This technique is particularly effective in crowded and cluttered environments where humans are close together. Also, the technique of the present disclosure is characterized by enhanced resolution providing for overcoming resolution limitations that make detecting closely-spaced humans challenging with traditional radar methods, and allows for distinguishing between people even when they're in proximity to each other.

Further, the technique of the present disclosure provides for reducing computational cost compared to traditional iterative algorithms. The unfolded network structure allows for faster processing once trained. The technique of the present disclosure provides for achieving higher localization precision by learning optimal parameters from data. The deep learning component enhances performance beyond what fixed-parameter iterative methods can achieve. The unfolding approach maintains the physical structure and unique features of the radar data.

The technique of the present disclosure also provides healthcare application value: It enables non-contact vital signs monitoring for multiple people simultaneously, which is particularly valuable for scenarios requiring contactless health monitoring (elderly care, hospital settings, pandemic situations). The technique of the present disclosure essentially bridges the gap between model-based signal processing (which has theoretical guarantees but limited adaptability) and data-driven deep learning (which is powerful but often lacks interpretability), offering the best of both approaches for the challenging problem of multi-person radar localization.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting examples only, with reference to the accompanying drawings, in which:

FIGS. 1A and 1B show schematically, by way of block diagrams, an exemplary radar-based localization system for use in the localization of one or more subjects according to the present disclosure;

FIGS. 2A to 2K show human thoracic motions (FIGS. 2A, 2B, 2E, 2F, 2I) and Discrete Fourier Transform (DFT) spectrum of the beamformed thoracic motion

(FIGS. 2C, 2D, 2G, 2H, 2K), wherein FIGS. 2A and 2C show results of human #1 at θ=0°, FIGS. 2B and 2D, 2E and 2G, 2F and 2H show estimates of human #1 for angle deviations Δθ: 0°, 10° and 20°, respectively, and FIGS. 21 and 2K show results of human #2 at θ=15°;

FIGS. 3A and 3B show block diagrams of the i'th iteration of RaLU-JSR algorithm (FIG. 3A) and the i'th layer of RaLU-Net (FIG. 3B) with the proposed modification;

FIGS. 4A to 4D show the detection accuracy of various methods according to various metrics and scores, wherein FIG. 4A shows the Average Recall (AR) metric,

FIG. 4B shows the Average Precision (AP) metric, FIG. 4C shows the F₁ score, and

FIG. 4D shows the average Mean Absolute Error (MAE) metric;

FIGS. 5A to 5F show the predicted localization maps of five compared methods for validation example #8 and angular deviation threshold (ADT) of 0° (FIGS. 5B to 5F) relative to the ground-truth (GT) localization map X̅_{GT} (FIG. 5A), wherein the tested methods are: angle-FFT (FIG. 5B), MUSIC-DoA (FIG. 5C), RaLU-JSR (I=15) (FIG. 5D), RaLU-JSR (I=100) (FIG. 5E), and RaLU-Net (I=15) (FIG. 5F); and

FIGS. 6A to 6F show the estimated angular pattern in the true range bin of the two adjacent humans (FIGS. 6B to 6F), compared to the GT angular positions (FIG. 6A).

### DETAILED DESCRIPTION OF EMBODIMENTS

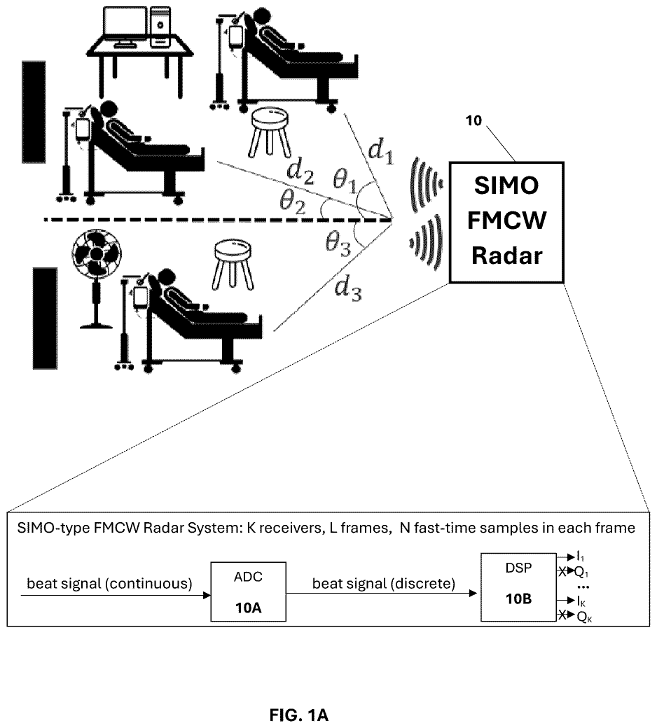

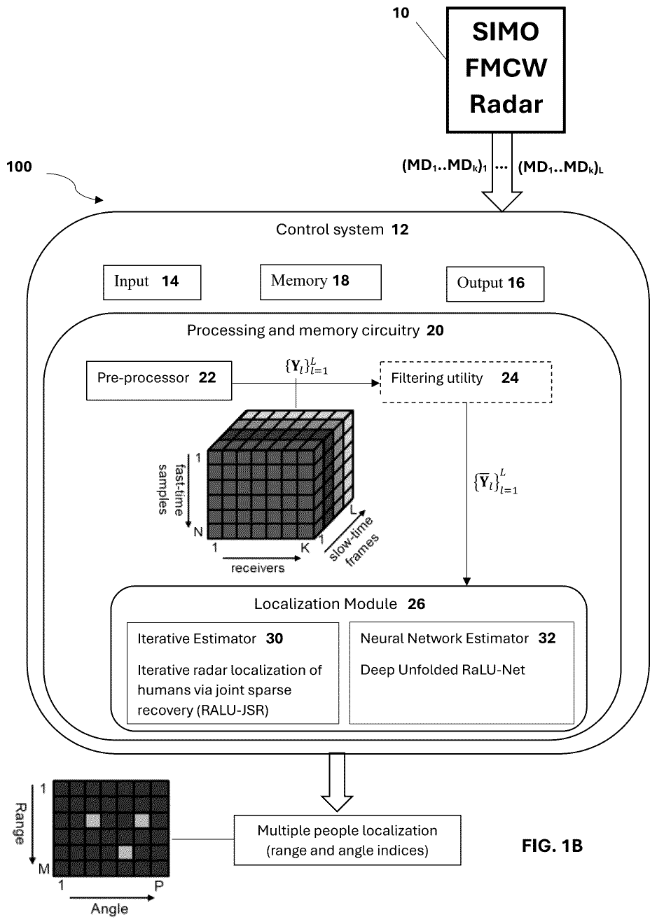

Reference is made to FIG. 1A illustrating an FMCW radar 10 in a SIMO (Single-Input Multiple-Output) configuration including a single transmitter and K (K>1) receivers. The configuration and operation of FMWC radar are generally known and need not be described in detail, except to note the following: FMCW radar continuously transmits a radio signal whose frequency changes over time, typically in a linear sweep (“chirp”). The transmitted signal's frequency increases or decreases steadily over a set period. When this signal reflects off a target and returns to the radar, there's a time delay. Because the frequency is constantly changing, the received signal has a different frequency than what is currently being transmitted. By measuring this frequency difference (“beat frequency”), the radar can determine the target's range.

The single transmitter emits L frames of chirp signals at a frame rate of fₛ, designed to localize Z≥1 humans within a cluttered environment containing U≥Z objects distributed across various angular and radial positions relative to the radar antennas. The present disclosure sets as a goal to determine the Z unknown human distances

$${\{ {d}^{( z )} \}}_{z = 1}^{Z}$$

and the Z unknown human angles

$${\{ {θ}^{( z )} \}}_{z = 1}^{Z} .$$

The Z≥1 humans need to be localized within a cluttered environment containing U≥Z objects distributed across various angular and radial positions relative to the radar antennas.

The k'th receiver captures the reflected echoes, which are separated into the I/Q channels, mixed with the transmitted signal, and sampled by an ADC 10A with interval T_{f} to produce discrete baseband (beat) signals of length N. The amplitudes of these signals are attenuated based on the radar cross-section (RCS) of reflecting objects. The use of In phase (I) and Quadrature (Q) channels of each receiver may be limited by imperfections such as non-orthogonality and channel gain mismatches which may reduce accuracy.

The inventors have already shown [4,5] that it is feasible to exploit only a single channel to address the above limitations. Therefore, in the present disclosure, raw measured data is used which is received from a single-channel (I or Q) front end of each receiver of the FMCW radar.

As mentioned above, in the present disclosure, reflected echoes of the radar are detected from U≥Z objects distributed across various angular and radial positions, where each object has a distinct pair of distance and angle {dᵤ, θₙ}, and the model of the present disclosure is formulated for M≥U general radial distances

$${\{ {d}_{m} \}}_{m = 1}^{M}$$

and P≥U general azimuth angles

$${\{ {θ}_{p} \}}_{p = 1}^{P} .$$

The Z unknown human distances

$${\{ {d}^{( z )} \}}_{z = 1}^{Z}$$

are, thus, included in the distances

$${\{ {d}_{m} \}}_{m = 1}^{M}$$

and the Z unknown human angles

$${\{ {θ}^{( z )} \}}_{z = 1}^{Z}$$

are among the angles

$${\{ {θ}_{p} \}}_{p = 1}^{P} .$$

Reference is made to FIG. 1B illustrating a radar-based localization system 100 of the present disclosure for use in localization of one or more subjects. The system 100 includes a control system 12 configured as a computerized system including, inter alia, an input 14, an output 16, a memory 18, and a processing and memory circuitry 20. The processing and memory circuitry 20 includes a preprocessor 22 and a localization module 26. In some embodiments, the processing and memory circuitry 20 includes also a filtering utility 24.

The control system 12 is configured for signal communication with the SIMO FMCW radar 10 to receive therefrom and process raw measured data. The raw measured data includes data pieces (MD₁, . . . , MD_{K})₁, . . . , (MD₁, . . . , MD_{K})_{L} being received from a single-channel front end of K receivers of the FMCW radar and corresponding to/being indicative of L measured data frames. Each MDₖ, for each frame l, includes N samples discretized by the ADC 10A and processed by the DSP 10B.

The first step of each data processing (i.e., localization) session involves preliminary processing of the radar channel data by the preprocessor 22 to construct a 3D cube,

$${\{ {Y}_{l} \}}_{l = 1}^{L}$$

satisfying the following novel bilinear model of the present disclosure:

Yₗ=AXᵢB+Wₗ,l=1, . . . ,L (1)

where the measured data is arranged into a matrix Yᵢ∈ [symbol] for each frame l.

A∈ [symbol] is a known range-related Vandermonde matrix, whose entries are given by A(n,m)≙e^{jω}^{ₘ}^{[n]}, where ωₘ is directly related to the radial distances

$${\{ {d}_{m} \}}_{m = 1}^{M} ,$$

as will be described in detail further below.

B∈ [symbol] is a known angle-related matrix whose entries are given by B(p,k)ϑe^{jφ}^{ₚ}^{[k]}, where

$${\{ {ϕ}_{p} [ k ] \}}_{k = 1}^{K}$$

are the phase shifts reflecting the azimuth angles

$${\{ {θ}_{p} \}}_{p = 1}^{P} ,$$

as will be described in detail further below.

Xₗ∈ [symbol] is an unknown matrix of complex amplitudes related to the radar cross section (RCS) of the reflecting objects, and
Wₗ∈ [symbol] is the noise matrix where Wₗ(n,k) is defined as zero mean i.i.d complex Gaussian noise.

The A and B matrices are assembled based on predefined frequency and angle grids, as will be described in more detail further below.

During continuous localization session, the FMCW radar periodically transmits and receives signals, and provides raw measured data corresponding to frames of chirp signals. This measured data is processed by the preprocessor 22 throughout the localization session. At each predefined time interval Tᵢₙₜ, the sequence

$${\{ {Y}_{l} \}}_{l = 1}^{L}$$

is constructed by aggregating all L frames recorded within the preceding time window T_{win}, where L is given by:

L=T_{win}fₛ.

The bilinear model of the present disclosure is assumed to exhibit the following properties:
A-1 The individuals being monitored remain stationary, with only slight thoracic movements due to cardiopulmonary activity. As a result, the {m,p} coordinates in matrices

$${\{ {X}_{l} \}}_{l = 1}^{L}$$

corresponding to their locations

$${\{ {d}^{( z )} , {θ}^{( z )} \}}_{z = 1}^{Z} ,$$

are fixed and joint across all L frames.
A-2 The number of objects within the radar's FOV, U, satisfies U<<MP, meaning that

$${\{ {X}_{l} \}}_{l = 1}^{L}$$

are U-sparse matrices.

Based on the bilinear model, the present disclosure provides for estimating the number of individuals, Z, along with their spatial locations

$${\{ {d}^{( z )} , {θ}^{( z )} \}}_{z = 1}^{Z} ,$$

by recovering the matrices

$${\{ {X}_{l} \}}_{l = 1}^{L} .$$

The assumptions A-1 and A-2 above mean that the U-sparse matrices

$${\{ {X}_{l} \}}_{l = 1}^{L}$$

share joint support. Here, the support is denoted by S and defined as the set of 2D {m, p} indices whose cardinality corresponds to the number of individuals Z and whose indices point to their respective range-angle locations

$${\{ {d}^{( z )} , {θ}^{( z )} \}}_{z = 1}^{Z}$$

and allow identifying the corresponding {m, p} indices.

Thus, the preprocessor (at times referred to also as preprocessor module) 22 is configured and operable to utilize the raw measured data (MD₁, . . . , MD_{K})₁, . . . , (MD₁, . . . , MD_{K})_{L} to create a data matrix Yₗ∈ [symbol] for each l-th frame, satisfying a bilinear model.

As will be described further below, the preprocessing module 22 may be configured and operable to utilize data indicative of predefined frequency and angle grids and assemble a range-associated matrix A and an angle-associated matrix B, satisfying a bilinear model: Yₗ=AXₗB+Nₗ, l=1, . . . , L. Here, Xₗ∈ [symbol] is the unknown matrix of complex amplitudes associated with reflections from various range-angle pairs, Mis a number of general radial distances, P is a number of general azimuth angles with respect to the radar, and Nₗ is a matrix defined by noise.

As mentioned above, in some embodiments, the control system 12 includes a filtering utility 24 configured and operable to suppress clutter by utilizing prior knowledge of human-typical pulse and breathing frequencies. Specifically, the filtering utility 24 extends the vital-based spectral filter, which has been described as being used for a single receiver [5], to the case of K>1 receivers. The data of each k'th antenna is selected by defining

Y^{(k)}∈ [symbol] :Y^{(k)}(n,l)≙Yₗ(n,k)

of Eq. (1) above.

Then, each filtered matrix is given by

$${\overline{Y}}^{( k )} = \frac{1}{L} {( {F}_{L}^{H} ( Π ⊙ {F}_{L} {Y}^{( k ) T} ) )}^{T} , k = 1 , … , K \qquad ( 2 )$$

where F_{L} is a full L-size discrete Fourier transform (DFT) matrix, Π∈ [symbol] denotes a window function corresponding to the vital frequencies in B^{(H)}∪B^{(R)} with B^{(R)} and B^{(H)}, respectively, denoting the frequency bands of respiration and heartbeat at rest, and ⊙ denotes the element-wise product.

The filtered data is then reshaped back to the original structure as in (1) denoted by

$${\{ {\overline{Y}}_{l} \}}_{l = 1}^{L} .$$

The localization module 26 is configured and operable to process each l-th data matrix Yₗ∈ [symbol] . This processing includes: iterative estimation of the localization of the one or more subjects by exploiting joint sparsity and cardiopulmonary properties of said data matrix; and/or utilizing a trained neural network with a predetermined number of layers to detect and localize the one or more subjects with angular resolution exceeding an angular resolution defined by the number K of receivers, and provide support recovery data indicative of range and angle indices of the one or more subjects.

Thus, the localization module 26 includes at least one of an iterative estimator 30 and a neural network (NN) estimator 32.

The iterative estimator 30 is configured and operable to perform an iterative estimation of the localization of the one or more subjects/humans by exploiting joint sparsity and cardiopulmonary properties of the measured data. The iterative approach is based on interpretation of multi-frame measured data by all-frame analysis, rather than frame-by-frame. More specifically, the algorithm comprises iterative minimization of the unknown matrix Xₗ:

$$\hat{𝕏} = {arg min}_{𝕏 ∈ {ℂ}^{M \times P \times L}} \frac{1}{2 L} {\sum }_{l = 1}^{L} {‖ {Y}_{l} - A {X}_{l} B ‖}_{F}^{2} + γ {‖ 𝕏 ‖}_{2 , 1} \qquad ( 3 )$$

using the joint sparsity of the localization matrices

$${\{ {X}_{l} \}}_{l = 1}^{L} ,$$

where ∥ [symbol] ∥_{2,1} refers to the sum of all l₂ norms over the frame (third) dimension of the 3D tensor [symbol] ∈ [symbol] which concatenates the frames of

$${\{ {X}_{l} \}}_{l = 1}^{L}$$

and γ≥0 is the regularization parameter.

The novel approach of the present disclosure extends the principles of a known JSR-FISTA (Joint Sparsity Recovery using Fast Iterative Shrinkage-Thresholding Algorithm) to the bilinear model of Eq. (1) above (referred to as RaLU-JSR method), by providing a soft-thresholding operator, as will be described in detail further below.

Another novel aspect of the RaLU-JSR algorithm of the present disclosure is that the joint sparsity across the L frames is utilized for the bilinear model of Eq. (1). The details of the joint sparsity utilization are described further below.

The neural network (NN) estimator 32 utilizes a trained neural network with a predetermined number of layers (iterations) to detect and localize the one or more subjects with angular resolution exceeding an angular resolution defined by the number K of receivers. As will be described further below, this technique is based on the new approach according to which neural network replaces the matrices A and B with weight matrices of appropriate sizes that will be learned from a training step.

In the following, the model for multi-person localization via SIMO FMCW is exemplified in detail. Based on this model, the novel iterative and unfolded human localization technique of the present disclosure is described. Further below, the performance of the algorithms of the present disclosure is assessed and compared with existing techniques.

Suppose an FMCW radar emits L frames of chirp signals within its FOV at a frame rate of fₛ, to localize and monitor the vital signs of Z≥1 humans. For each frame, the reflected echo signals are mixed with the transmitted ones and sampled by an ADC, yielding length-N discrete base-band (beat) signals [12], [13] with attenuated amplitudes corresponding to the radar-cross-section (RCS) of the measured objects. The inventors consider a SIMO Uniform Linear Array (ULA) with a single transmitter and K receivers located at rₖ≙(k−1)λ/2, for k=1, . . . , K, where λ is the chirp's maximal wavelength.

To formulate the problem of localizing and monitoring the vital signs of multiple humans using a SIMO FMCW radar, a bilinear model is adopted, suggested in [4] for M≤N general radial distances

$${\{ {d}_{m} \}}_{m = 1}^{M}$$

and P≥K general azimuth angles

$${\{ {θ}_{p} \}}_{p = 1}^{P} :$$

where Yₗ∈ [symbol] is the input data matrix, A∈ [symbol] is the range-related Vandermonde dictionary matrix, whose entries are given by A(n,m)≙e^{j2πf}^{ₘ}^{nT}^{_{f}} where T_{f} denotes the fast-time sampling interval determined by the ADC component, and

$${\{ {f}_{m} \}}_{m = 1}^{M}$$

are distinct beat frequencies proportional to

$${\{ {d}_{m} \}}_{m = 1}^{M}$$

by

$${f}_{m} {=}^{Δ} \frac{2 S}{c} {d}_{m} , m = 1 , … , M \qquad ( 5 )$$

where c denotes the speed of light and S≙B/T_{c} is the rate of the frequency sweep of each chirp, with B and T_{c} respectively being the chirp's total bandwidth and duration. The unknown human distances, denoted by

$${\{ {d}^{( z )} \}}_{z = 1}^{Z} ,$$

are among the set

$${\{ {d}_{m} \}}_{m = 1}^{M} .$$

B∈ [symbol] is the angle-related dictionary matrix, whose entries are given by B(p,k)≙e^{jφ}^{ₚ}^{[k]} where

$${\{ {ϕ}_{p} [ k ] \}}_{p = 1}^{P}$$

are phase shifts due to the ULA geometry:

$${ϕ}_{p} [ k ] = \frac{2 π}{λ} {r}_{k} sin {θ}_{p} , p = 1 , … , P \qquad ( 6 )$$

where the unknown human angles, denoted by

$${\{ {θ}^{( z )} \}}_{z = 1}^{Z}$$

are among the angle set

$${\{ {θ}_{p} \}}_{p = 1}^{P} .$$

Xₗ∈ [symbol] is the unknown matrix of complex amplitudes associated with reflections from various range-angle pairs.

Specifically, Xₗ(m,p)≙x_{m,p}e^{jψ}^{_{m,p}}^{[l]} with slow-time varying phase term

$${ψ}_{m , p} [ l ] {=}^{Δ} \frac{4 π}{λ} ( {d}_{m} + {v}_{m , p} [ l ] )$$

where the discrete vibration v_{m,p}[l] can be modeled by a sum of cosines [4], [5] to represent both static and vibrating objects. The human thoracic vibrations denoted by

$${\{ {v}^{( z )} [ l ] \}}_{z = 1}^{Z} ,$$

belong to the set

$${\{ {v}_{m , p} [ l ] \}}_{l = 1}^{L}$$

and contain the information of the corresponding vital signs. Lastly, the elements of Nₗ∈ [symbol] are derived from a zero mean i.i.d. complex Gaussian noise with variance σ². It is noted that the model in Eq. (4) above is adequate for typical indoor conditions, where the received signal is influenced by U≥Z objects at various angular and radial positions.

To localize the position of the humans given L_{loc} data frames

$${\{ {Y}_{l} \}}_{l = 1}^{{L}_{loc}} ,$$

the following assumptions are made on the model in Eq. (4):
A-1: Each monitored person is stationary except for small thoracic movements caused by breathing.
A-2: Consider U≥Z≥1 objects in the radar's FOV. Then, U<<MP meaning that

$${\{ {X}_{l} \}}_{l = 1}^{L}$$

are U-sparse matrices.

Based on the model in Eq. (4), the goal is to estimate the number of humans Z and their polar coordinates

$${\{ {d}^{( z )} , {θ}^{( z )} \}}_{z = 1}^{Z}$$

which by Eq. (5) and (6) are reflected in proper {m, p} indexes of

$${\{ {X}_{l} \}}_{l = 1}^{L} .$$

In the following, the iterative and deep unfolding strategies for radar-based human localization are described. The inventors first provide an iterative method that extends the (fast) iterative soft thresholding algorithm, (F)ISTA [14], [15] for the bilinear model in Eq. (4) which capitalizes on joint sparsity across frames and cardiopulmonary properties. Then, an unfolded network is proposed, based on the latter to enhance accuracy and computational efficiency, when training data is available.

### A. Iterative RaLU-JSR

Consider L input data frames

$${\{ {Y}_{l} \}}_{l = 1}^{L}$$

satisfying the model in Eq. (4). Then, since based on assumptions A-1 and A-2, the localization matrices

$${\{ {X}_{l} \}}_{l = 1}^{L}$$

share joint support,

$${\{ {X}_{l} \}}_{l = 1}^{L}$$

is estimated by promoting the joint sparsity via the following 3D l_{2,1}-norm regularized Least-Squares (LS) problem

$$\hat{𝕏} = {arg min}_{𝕏 ∈ {ℂ}^{M \times P \times L}} \frac{1}{2 L} {\sum }_{l = 1}^{L} {‖ {Y}_{l} - {AX}_{l} B ‖}_{F}^{2} + γ {‖ 𝕏 ‖}_{2 , 1} \qquad ( 7 )$$

where ∥ [symbol] ∥_{2,1} refers to the sum of all l₂ norms over the frame (third) dimension of the 3D tensor [symbol] ∈ [symbol] which concatenates the frames of

$${\{ {X}_{l} \}}_{l = 1}^{L} .$$

To solve Eq. (7), the well-known (F)ISTA algorithm [14], [15] is extended for the given bilinear case of Eq. (4), which is referred to herein as RaLU-JSR: Radar Localization of hUmans via Joint Sparse Recovery. The general step is given by:

$${\{ {X}_{l}^{( i + 1 )} \}}_{l = 1}^{L} = {𝒯}_{\frac{γ}{{L}_{f}}}^{( 3 D )} ( {\{ {G}_{l}^{( i )} \}}_{l = 1}^{L} ) , i = 1 , ... , I \qquad ( 8 )$$

where I denotes the maximal number of iterations, γ is the regularization parameter, L_{f} denotes (F)ISTA's Lipschitz constant [14], here set to L_{f}=λₘₐₓ(A^{H}A)λₘₐₓ(B^{H}B) with λₘₐₓ denoting the largest singular value. Lastly,

$${𝒯}_{α}^{( 3 D )} ( \cdot )$$

is the 3D soft-threshold operator which makes each {m,p}'th element of

$${X}_{l}^{( i + 1 )}$$

l=1, . . . , L equal to

$${X}_{l}^{( i + 1 )} ( m , p ) = max ( 0 , 1 - α / {‖ {𝔾}^{( i )} ( m , p ) ‖}_{2} ) \cdot {G}_{l}^{( i )} ( m , p ) ,$$

where [symbol] ∈ [symbol] C concatenates the frames of

$${\{ {G}_{l} \}}_{l = 1}^{L} , {G}_{l} {=}^{Δ} {X}_{l} - \frac{1}{{L}_{f}} {g}_{l} ( {X}_{l} )$$

with gradient:

gₗ(Xₗ)≙A^{H}(AXₗB−Yₗ)B^{H}.

It is noted that by placing gₗ(X) in Gₗ, one obtains:

$${G}_{l} = \frac{1}{{L}_{f}} {A}^{H} {Y}_{l} {B}^{H} - \frac{1}{{L}_{f}} {A}^{H} {AX}_{l} {BB}^{H} + {X}_{l} .$$

The proposed iterative RaLU-JSR algorithm for minimizing Eq. (7) requires knowledge of A and B, which can be obtained by establishing appropriate distance and angle grids for Eq. (5) and (6), respectively. In addition, one can reduce the influence of non-human objects by filtering

$${\{ {Y}_{l} \}}_{l = 1}^{L}$$

according to the slow-time filter introduced in [5] for each receiver's channel data with Π∈ [symbol] denoting the filter's window function corresponding to cardiopulmonary frequencies at rest. The filtered set is denoted by

$${\{ {\overline{Y}}_{l} \}}_{l = 1}^{L} .$$

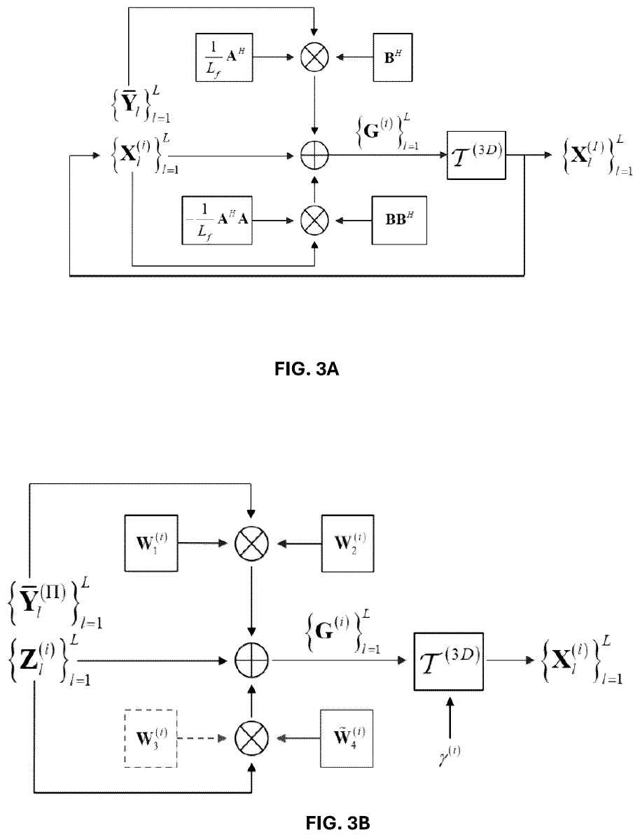

Algorithm 1 summarizes the RaLU-JSR algorithm, and FIG. 3A depicts it in a block diagram. It is noted that the convergence rate can be accelerated by using FISTA's update step of linearly combining the previous two estimates [14], i.e., replacing

$${X}_{l}^{( i + 1 )} with {Z}_{l}^{( i + 1 )} = {X}_{l}^{( i )} + \frac{{t}^{( i )} - 1}{{t}^{( i + 1 )}} ( {X}_{i}^{( i )} - {X}_{l}^{( i - 1 )} )$$

where:

t⁽ⁱ⁺¹⁾=0.5(1+\sqrt{1+4t⁽ⁱ⁾²}).

Algorithm 1: RaLU-JSR for minimizing Eq. (7)

$Input : {\{ {\overline{Y}}_{l} \}}_{l = 1}^{L} , A , B , {L}_{f} , γ > 0 , I$
$Initialize : i = 1 , \{ {X}_{l}^{( 0 )} = {0}^{M \times P} , l = 1 , … , L \}$
while i < I or stopping criteria not fulfilled do
$1 : {G}_{l}^{( i )} = \frac{1}{{L}_{f}} {A}^{H} {\overline{Y}}_{l} {B}^{H} - \frac{1}{{L}_{l}} {A}^{H} A {X}_{l}^{( i )} B {B}^{H} + {X}_{l}^{( i )} , l = 1 , … , L$
$2 : {\{ {X}_{l}^{( i + 1 )} \}}_{l = 1}^{L} = {𝒯}_{{L}_{l}^{′}}^{( 3 D )} ( {\{ {G}_{l}^{( i )} \}}_{l = 1}^{L} )$
3: i + i + 1
end while
$Return : {\{ {X}_{l}^{( I )} \}}_{l = 1}^{L}$

The support, denoted by [symbol] , relates to the number of humans by its cardinality and to their location by the range-angle indices it contains. [symbol] is determined by first using the average power over the frames of

$${\{ {X}_{l}^{( l )} \}}_{l = 1}^{L}$$

which results in a 2 D range-angle localization map X̅∈ [symbol] . Then, various methods can be used to evaluate [symbol] from X, such as CA-CFAR or 2D peak-detection [4]. In numerous radar systems, the potential angular resolution is compromised by a limited number of receiving antennas [2]-[4], hence posing challenges in accurate separation and positioning, particularly in scenarios involving close proximity between individuals. Thus, the inventors unfold ReLU-JSR to further enhance accuracy and super-resolution capabilities while reducing computational cost, provided that credible training data is available.

### B. Deep Unfolded RaLU-Net

Generally, this novel RaLU-NET technique of the present disclosure combines the principles of iterative joint sparsity recovery algorithms (i.e., optimization exploiting the shared sparse structure in multi-person radar data) with deep unfolding approach (i.e., the iterative algorithm is “unrolled” into neural network layers, where each iteration becomes a layer with learnable parameters) and radar localization for SIMO FMCW.

Here, the inventors provide viewing the i'th iteration of Algorithm 1 as the i'th layer in a model-based network [16], by replacing the matrices depending on A and B with weight matrices of appropriate sizes that will be learned from training data. The i'th layer of the network can be described by:

$$\{ \begin{matrix}{G}_{l}^{( i )} = {W}_{1}^{( i )} {\overline{Y}}_{l} {W}_{2}^{( i )} + {W}_{3}^{( i )} {X}_{l}^{( i )} {W}_{4}^{( i )} + {X}_{l}^{( i )} , l = 1 , … , L \\ {\{ {X}_{l}^{( i + 1 )} \}}_{l = 1}^{L} = {𝒥}_{{γ}^{( i )}}^{( 3 D )} ( {\{ {G}_{l}^{( i )} \}}_{l = 1}^{L}\end{matrix} ) \qquad ( 9 )$$

with learnable

$$Θ = {\{ {W}_{1}^{( i )} , {W}_{2}^{( i )} , {W}_{3}^{( i )} , {W}_{4}^{( i )} , {γ}^{( i )} \}}_{i = 1}^{I}$$

for I layers.

Since the present disclosure resolves the problem of human localization for enhanced multi-person NCVSM, the network is modified accordingly, as shown in the steps below.

First, the weights are initialized in accordance with the multiplications in RaLU-JSR, i.e.,

$${W}_{1}^{( i )} = \frac{1}{{L}_{f}} {A}^{H} , {W}_{2}^{( i )} = {B}^{H} , {W}_{3}^{( i )} = - \frac{1}{{L}_{f}} {A}^{H} A and {W}_{4}^{( i )} = {BB}^{H} .$$

To assemble A, it is assumed that the frequencies in Eq. (5) lie on the Nyquist grid:

$${f}_{m} = \frac{{f}_{ADC}}{N} {i}_{m}$$

for iₘ=0, . . . , M−1 where f_{ADC}≙1/T_{f} is determined by the ADC component and M=N/2 to consider only the non-negative spectrum.

To assemble B, the angles in Eq. (6) are set to cover a FOV of 180 degrees, i.e.,
θₚ=−90+iₚ for iₚ=0, . . . , P−1, where P=180.

It should be noted that now

$$A ( n , m ) = {e}^{j 2 π \frac{{i}_{m}}{N} n} ,$$

which means that A^{H}A=NI_{M} with I_{M} denoting a size-M identity matrix.

Since

$${W}_{3}^{( i )} = - \frac{1}{{L}_{f}} {A}^{H} A = - \frac{N}{{L}_{f}} {I}_{M} ,$$

the inventors choose to remove

$${W}_{3}^{( i )}$$

from the network and reinitialize

$${W}_{4}^{( i )} by {\tilde{W}}_{4}^{( i )} = - \frac{N}{{L}_{f}} {BB}^{H} .$$

Second, similarly to the RaLU-JSR, the inventors filter the input set

$${\{ {Y}_{l} \}}_{l = 1}^{L}$$

to mitigate the effect of non-human clutter, only here the window function Π is learned through the training process and initialized with the vital band B^{(v)} corresponding to cardiopulmonary frequencies at rest. The l'th filtered input is denoted by

$${\overline{Y}}_{l}^{( \prod )} .$$

Third, FISTA's accelerating update step [14] is adopted for each layer with learned momentum parameter ρ⁽ⁱ⁾ [2917] and learned regularization parameter γ⁽ⁱ⁾ initialized by ρ and γ, respectively, similarly to RaLU-JSR.

To conclude, the i'th layer of the modified network, termed herein RaLU-Net, can be expressed by:

$$\{ \begin{matrix}{G}_{l}^{( i )} = {W}_{1}^{( i )} {\overline{Y}}_{l}^{( \prod )} {W}_{2}^{( i )} + {Z}_{l}^{( i )} {W}_{4}^{( i )} + {Z}_{l}^{( i )} , l = 1 , … , L \\ {\{ {X}_{l}^{( i )} \}}_{l = 1}^{L} = {{𝒥}_{{γ}^{( i )}}^{( 3 D )} ( {\{ {G}_{l}^{( i )} \}}_{l = 1}^{L} )}^{1} \\ {Z}_{l}^{( i + 1 )} = {X}_{l}^{( i )} + {ρ}^{( i )} ( {X}_{i}^{( i )} - {X}_{l}^{( i - 1 )} ) , l = 1 , … , L ,\end{matrix} \qquad ( 10 )$$

where the average power operator is added after the l'th layer to obtain the predicted localization map X̅.

Here,

$$Θ = \{ {\{ {W}_{1}^{( i )} , {W}_{2}^{( i )} , {W}_{3}^{( i )} , {W}_{4}^{( i )} , {γ}^{( i )} , {ρ}^{( i )} \}}_{i = 1}^{I} , Π \}$$

and a block diagram of the i'th layer is given in FIG. 3B.

RaLU-Net is trained using back-propagation in a supervised manner given Nₜᵣ training examples of

$${\{ {Y}_{l} \}}_{l = 1}^{L}$$

and corresponding ground-truth (GT) localization maps X̅_{GT} containing the expected magnitudes given only humans in the FOV.

Since X̅_{GT} is a sparse matrix (A-2), a mask loss is used between the predicted map X̅ and the true map X̅_{GT} which comprised of the Mean Squared Error (MSE) loss in the support [symbol] with l₁ regularization over the complementary set [symbol] :

$${ℒ}_{m a s k} ( \overline{X} , {\overline{X}}_{G T} ) = {ℒ}_{M S E}^{( 𝒮 )} + {μℒ}_{{l}_{1}}^{( {𝒮}^{𝒸} )} where {ℒ}_{M S E}^{( 𝒮 )} = \frac{1}{\vert 𝒮 \vert} {\sum }_{\{ m , p \} ∈ 𝒮} {( \overline{X} ( m , p ) - {\overline{X}}_{G T} ( m , p ) )}^{2} , {ℒ}_{{l}_{1}}^{( {𝒮}^{𝒸} )} = \frac{1}{\vert {𝒮}^{𝒸} \vert} {\sum }_{\{ m , p \} ∈ {𝒮}^{𝒸}} \vert \overline{X} ( m , p ) \vert \qquad ( 11 )$$

and μ balances between these objectives. for Nₜᵣ training pairs, the total loss of the network is:

$$ℒ ( Θ ) = \frac{1}{{N}_{t r}} {\sum }_{{n}_{t r} = 1}^{{N}_{t r}} {ℒ}_{m a s k} ( f ( {\{ {Y}_{l , {n}_{t r}} \}}_{l = 1}^{ℒ} , Θ ) , {\overline{X}}_{GT , {n}_{tr}} ) \qquad ( 12 )$$

where f(·, Θ) is the output of the network.

In the following, the performance of the technique of the present disclosure is evaluated and compared to the existing techniques through a simulation based on the model in Eq. (4) that involves real-world data from [10]. First, the experimental setup is described and then the results of the application of the model to localize multiple individuals are presented.

The simulated data was established as follows. First, 10000 sets of

$${\{ {Y}_{l} \}}_{l = 1}^{L}$$

corresponding to Eq. (4) were generated, of which Nₜᵣ=8000 were allocated for training RaLU-Net and Nᵥₗ=2000 for validating all compared methods. Each set reflects the FOV of U=7 distinct objects including Z=3 humans, 2 static entities, and 2 vibrating fans. W.r.t. the radar antennas, the objects were randomly positioned within a range and angle of 0.15 to 4 meters and −40 to 40 degrees, respectively, with the constraint that two out of the three individuals exhibit an angular difference of 15 degrees. It is noted that this is half of the theoretical angle resolution [6] for the usage of 4 receiving antennas, which is addressed in this disclosure. The amplitudes of the reflective wave from the human, clutter, and fan were set to 0.9, 1 and 0.8, respectively. The human thoracic vibrations

$${\{ {v}^{( z )} [ l ] \}}_{z = 1}^{Z}$$

were based on the impedance signals of subjects 1-3 from [10]. The static clutter has no vibration and the fan's vibration was modeled by a_{fan} cos(2πf_{fin}lTₛ) with a_{fan}=10⁻³ and f_{fan}=20 [Hz][7].

The FMCW parameters for assembling the model in Eq. (4) were set as follows: λ=3.9 [mm], T_{c}=57[μs], S=70 [MHz/μs], f_{ADC}=4 [MHz] with selected N=200 fast-time samples, K=4 receivers and L=100 slow-time frames of length Tₛ=50 [ms](fₛ=20 [Hz]), corresponding to a transmission time of 5 seconds. It is noted that although continuous vital signs monitoring normally lasts between a few minutes to several hours, the time required for the localization step can be up to a few seconds only [4], [5]. To simulate environmental noise, the variance of the Gaussian elements of Nₗ(1) was set according to SNR≙1/σ₂=−2 [dB].

The inventors assessed the detection and positioning performance of the iterative RaLU-JSR and unfolded RaLU-Net methods of the present disclosure through all Nᵥₗ validation examples, and compared them to the AngleFFT method [2], [3], [6] and to the super-resolution MUSIC-DOA approach [7], [8]. The number of iterations in RaLU-JSR was set to both I=15 and I=100 while the number of layers in RaLUNet was set to I=15.

The initialization of RaLU-JSR is equivalent to that of RaLU-Net discussed above in subsection Deep Unfolded RaLU-Net for A, B, L_{f}, γ=60, ρ=1 and B^{(v)}=[0.1 1.67][Hz]. RaLU-Net employed a batch size of 8 and the ADAM optimizer with a learning rate of 10⁻⁶ and μ=0.5 (8). For a fair comparison, the maps of all methods were produced using the same range and angle grids and were normalized according to each map's maximum value. Finally, the support S was determined by 2D selection of peaks with a power of at least 0.75. Here, the inventors determined that a person is detected if the estimated range and angle w.r.t. the human's true location is within 4 [cm] (≈range-bin) and within angular deviation threshold (ADT) of the following degrees: 0, 1, 5, 10, 15 and 20. Based on these criteria, the Average Recall (AR) and Average Precision (AP) metrics [18] were employed as well as the F₁ score [19] which combines both AR and AP into a single metric. As for the positioning error, the average Mean Absolute Error (MAE) metric was used for each detected human.

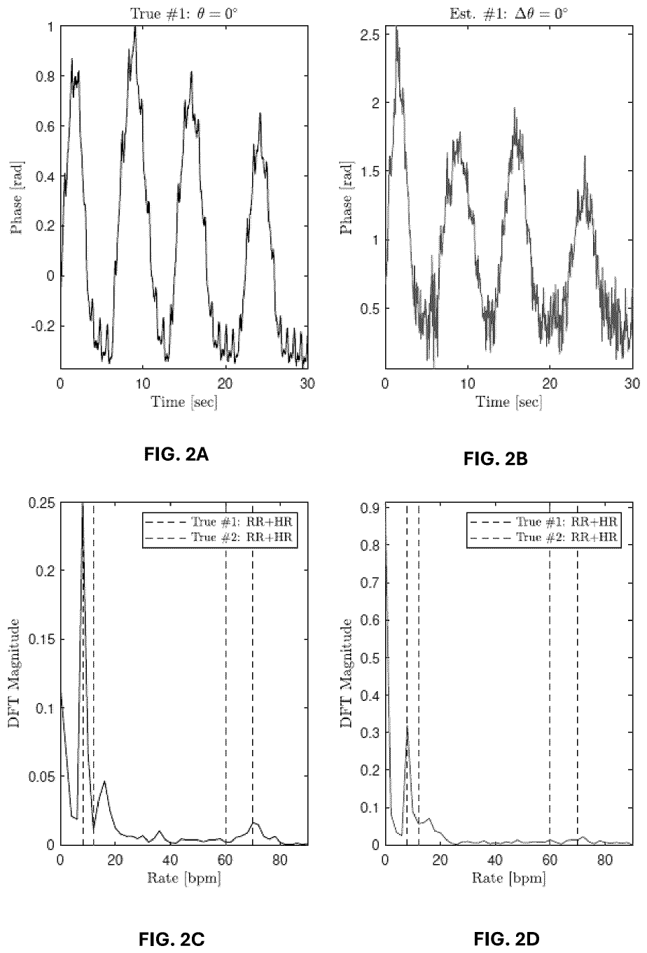

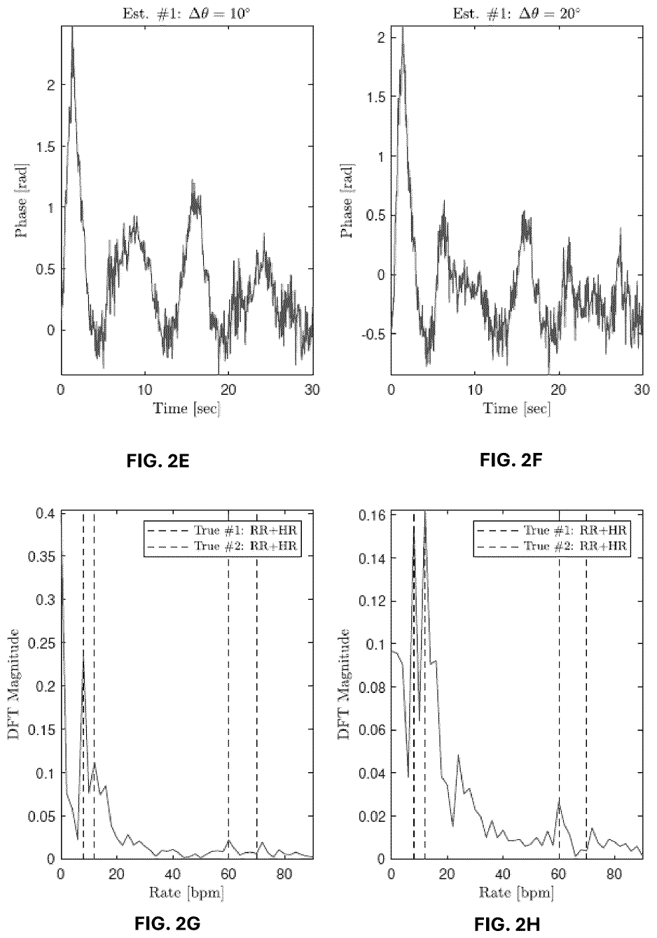

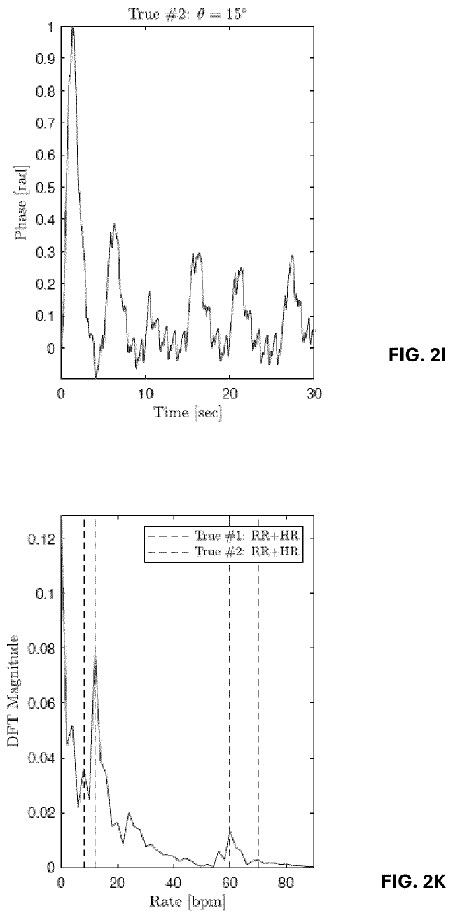

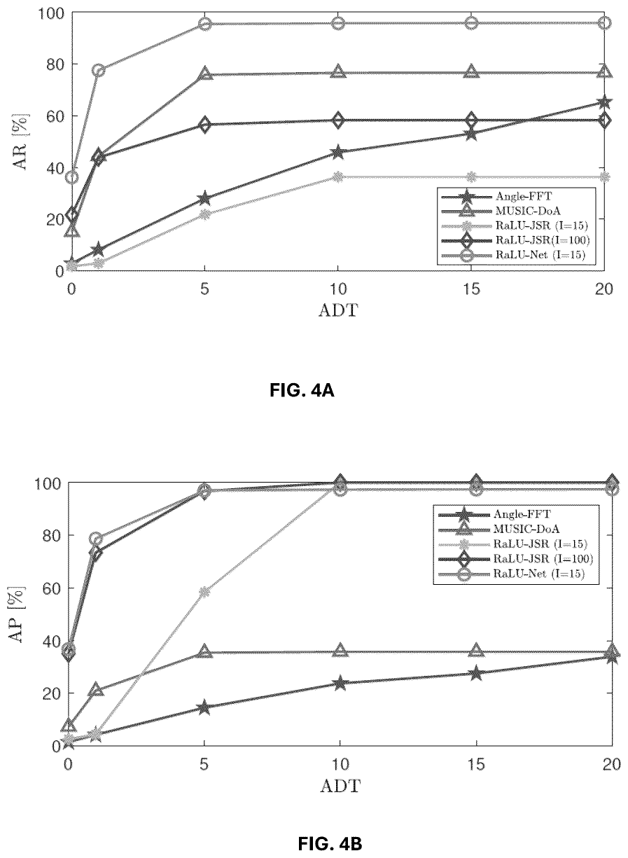

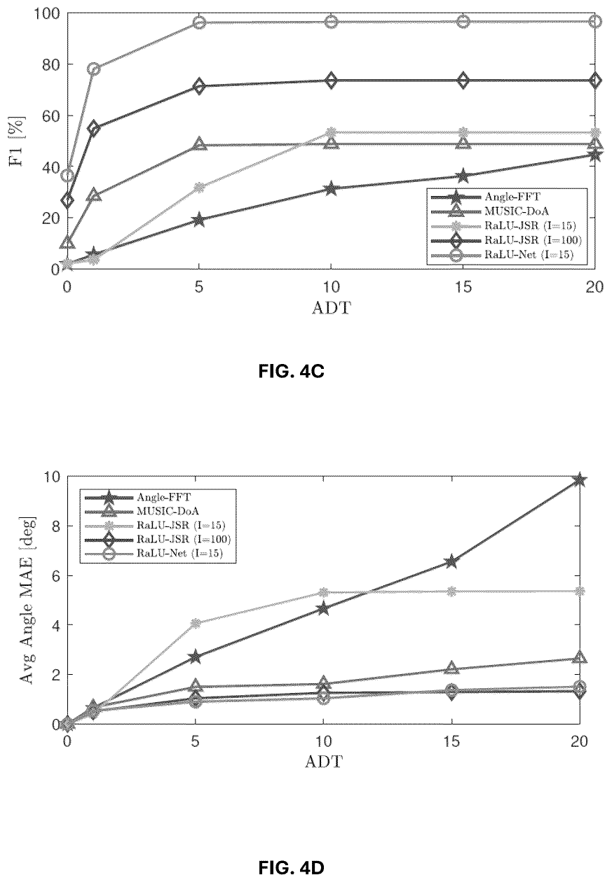

FIGS. 4A to 4D show the average scores for the entire validation set vs. ADT of all compared methods. First, one can observe that the F₁ score of the RaLU-Net outperforms all other methods with a particularly high score of over 96% for any ADT above 5°, which is a particularly stringent condition when considering a theoretical resolution of 30°. The iterative RaLU-JSR for I=100 and I=15 achieved a high AP score comparable to RaLU-Net for ADT above 5° and 10°, respectively. However, this came at the expense of low AR due to the missing detection of humans. The MUSIC-DOA technique seeks up to K/2 strongly oscillating objects in each range-bin. This is indicative of that for true range bins, the angle estimations and separation abilities are adequate (high AR), yet this yields a large number of false detections (low AP). The main reason for the weak overall detection performance of Angle-FFT stems from the high sensitivity to clutter in the FOV and to the theoretical angle resolution limitation. As for the positioning (bottom right), it is apparent that both RaLuNet and RaLU-JSR achieved a minimal average angle MAE of <1.5° for every ADT which is essential for accurate NCVSM, as can be seen in FIGS. 2A to 2K. However, whereas the accelerated iterative method requires 100 iterations, the unfolded network employs only 15 layers, which is significantly lighter computationally. All approaches perform well in terms of average range MAE of <2 [cm] due to a high-frequency ADC sampler and large bandwidth of 4 [GHz]. Hence, this evaluation is omitted.

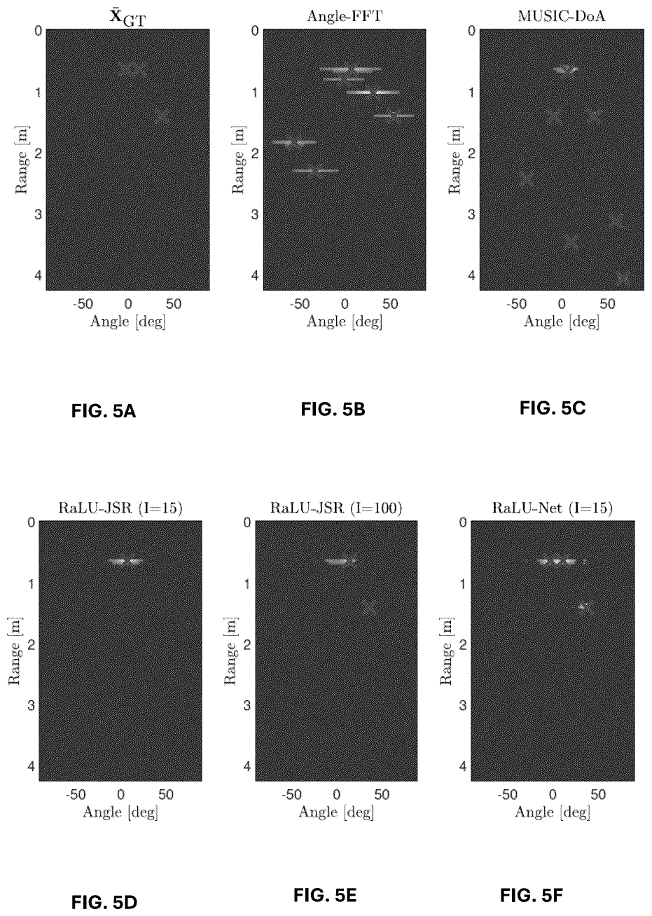

FIGS. 5A to 5F depict the predicted localization maps of all 5 compared methods for validation example #8 and ADT=5° relative to X̅_{GT} in the leftmost column. First, one can observe that only RaLU-Net detects exactly 3 out of 3 humans without any misses or false detections.

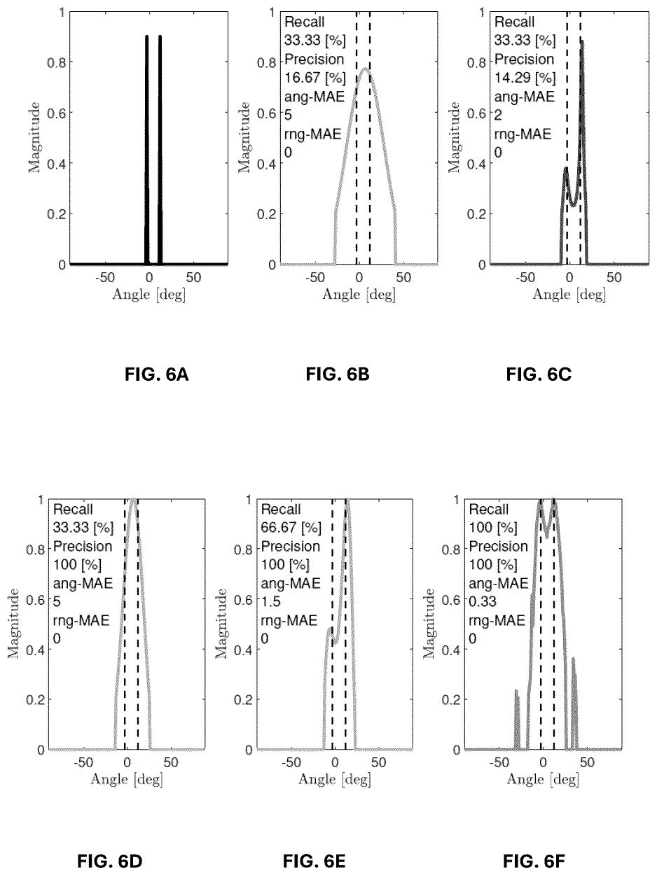

FIGS. 6A to 6F show the estimated angular pattern in the true range bin of the two adjacent humans, compared to the GT angular positions (leftmost figure). While for the iterative RaLU-JSR, the more iterations, the better the performance, RaLUNet exhibits the most distinct separation by both power and shape, as well as angular positioning with the lowest Average Angle MAE compared to all other techniques.

Thus, in the present disclosure, the iterative RaLU-JSR method is provided that, by leveraging joint sparsity in the data along with human cardiopulmonary properties, enhances the localization of multiple individuals, even in close proximity and a cluttered environment, for precise multi-person NCVSM via SIMO FMCW radar. Then, the inventors unfold RaLU-JSR into a neural network called RaLU-Net, which further optimizes performance and computational efficiency, given the availability of high-quality training data. Simulation studies involving real-world data show remarkable results on 2000 distinct scenarios via key metrics for detection and positioning. The novel methods of the present disclosure offer valuable opportunities for developing advanced human localization and NCVSM systems in cluttered scenarios where accuracy and efficiency are critical.

1. A radar-based localization system for use in localization of one or more subjects, the radar-based localization system comprising a control system configured for signal communication with a single-input multiple-output (SIMO) frequency modulated continuous wave (FMCW) radar to process raw measured data which is received from a single-channel front end of each of K receivers (K>1) of said SIMO FMCW radar and which is indicative of L measured data frames, each comprising N consecutive beat signals, said control system being configured and operable as a computerized system comprising data input and output utilities, memory utility and a data processing utility, said data processing utility comprising:
a preprocessing module configured and operable to utilize said raw measured data to create, for each l-th frame, a data matrix Yₗ∈ [symbol] satisfying a bilinear model; and
a localization module configured and operable to process each said l-th data matrix Yₗ∈ [symbol] by carrying out at least one of the following:
(i) iterative estimation of the localization of the one or more subjects by exploiting joint sparsity and cardiopulmonary properties of said data matrix, and
(ii) utilizing a trained neural network to detect and localize the one or more subjects with angular resolution exceeding an angular resolution defined by the number K of receivers, and
provide support recovery data indicative of range and angle indices of the one or more subjects.

2. The radar-based localization system of claim 1, wherein said preprocessing module is configured and operable to utilize data indicative of predefined frequency and angle grids and assemble a range-associated matrix A and an angle-associated matrix B, satisfying a bilinear model: Yₗ=AXₗB+Nₗ, l=1, . . . , L, where Xₗ∈ [symbol] is an unknown matrix of complex amplitudes associated with reflections from various range-angle pairs, M being a number of general radial distances, P being a number of general azimuth angles with respect to the radar, and Nₗ is a matrix defined by noise.

3. The radar-based localization system of claim 1, wherein said data processing utility comprises a filtering utility configured and operable to utilize prior knowledge of human typical pulse and breathing frequencies to define a filter applied on an input set of said data matrices,
$${\{ {Y}_{l} \}}_{l = 1}^{L} ,$$
to reduce non-human clutter.

4. The radar-based localization system of claim 2, wherein said iterative estimation of the localization of the one or more subjects by exploiting joint sparsity and cardiopulmonary properties of said data matrix utilizes a fast iterative soft-thresholding (FISTA) algorithm to perform the following minimization:
$$\hat{𝕏} = {arg min}_{𝕏 ∈ {ℂ}^{M \times P \times L}} \frac{1}{2 L} {\sum }_{l = 1}^{L} {‖ {Y}_{l} - A {X}_{l} B ‖}_{F}^{2} + γ {‖ 𝕏 ‖}_{2 , 1}$$
where ∥ [symbol] ∥_{2,1} refers to the sum of all l₂ norms over the frame dimension of a 3D tensor [symbol] ∈ [symbol] which concatenates the frames of
$${\{ {X}_{l} \}}_{l = 1}^{L}$$
and γ≥0 is a regularization parameter, said minimization providing the unknown set
$${\{ {X}_{l} \}}_{l = 1}^{L} .$$

5. The radar-based localization system of claim 4, wherein said iterative estimation of the localization is accelerated by using an update step where two previous estimates are linearly combined replacing
$${X}_{l}^{( i + 1 )} by {Z}_{l}^{( i + 1 )} = {X}_{l}^{( i )} + \frac{{t}^{( i )} - 1}{{t}^{( i + 1 )}} ( {X}_{l}^{( i )} - {X}_{l}^{( i - 1 )} )$$
where t⁽ⁱ⁺¹⁾=0.5 (1+\sqrt{1+4t⁽ⁱ⁾²}) initialized to t=0.

6. The radar-based localization system of claim 4, wherein said support recovery data indicative of range and angle indices of the one or more subjects is estimated by determining an average power across the frames of
$${\{ {X}_{l} \}}_{l = 1}^{L}$$
resulting in a 2D range-angle localization map.

7. The radar-based localization system of claim 2, wherein said processing of each said l-th data matrix Yₗ∈ [symbol] by utilizing the trained neural network with the predetermined number of layers comprises replacing the matrices A and B with weight matrices {Wᵢ} of appropriate sizes, said weight matrices being learned from train data.

8. The radar-based localization system of claim 7, wherein said weight matrices {Wᵢ} of appropriate sizes comprise a set
$${\{ {W}_{1}^{( i )} , {W}_{2}^{( i )} , {W}_{3}^{( i )} , {W}_{4}^{( i )} \}}_{i = 1}^{I} ,$$
I being the number of layers, wherein said weight matrices {Wᵢ} are initialized to:
$${W}_{1}^{( i )} = \frac{1}{{L}_{f}} {A}^{H} , {W}_{2}^{( i )} = {B}^{H} , {W}_{3}^{( i )} = - \frac{1}{{L}_{f}} {A}^{H} A , and {W}_{4}^{( i )} = B {B}^{H} ,$$
where L_{f} is a constant set to L_{f}=λₘₐₓ(A^{H}A)λₘₐₓ(B^{H}B) with λₘₐₓ denoting the largest singular value.

9. The radar-based localization system of claim 8, wherein said matrix
$${W}_{3}^{( i )}$$
is omitted and said matrix
$${W}_{4}^{( i )}$$
is reinitialized to
$${\tilde{W}}_{4}^{( i )} = - \frac{N}{{L}_{f}} {BB}^{H} .$$

10. The radar-based localization system of claim 9, wherein said data processing utility comprises a filtering utility configured and operable to apply a filter on an input set of said data matrices
$${\{ {Y}_{l} \}}_{l = 1}^{L}$$
to reduce non-human clutter, said filter being learned from said training data and initialized utilizing prior knowledge on cardiopulmonary frequencies at rest.

11. A computer-implemented method for localization of one or more subjects, the method comprising:
obtaining raw measured data corresponding to data being received from a single-channel front end of each of K receivers (K>1) of a SIMO FMCW radar and being indicative of L measured data frames, each comprising N consecutive beat signals;
utilizing said raw measured data to create, for each l-th frame, a data matrix Yₗ∈ [symbol] satisfying a bilinear model; and
performing preprocessing of each said l-th data matrix Yₗ∈ [symbol] by carrying out at least one of the following:
(i) iterative estimation of the localization of the one or more subjects by exploiting joint sparsity and cardiopulmonary properties of said data matrix, and
(ii) utilizing a trained neural network to detect and localize the one or more subjects with angular resolution exceeding an angular resolution defined by the K receivers; and
generating support recovery data indicative of range and angle indices of the one or more subjects.

12. The method of claim 11, wherein said preprocessing comprises utilizing data indicative of predefined frequency and angle grids and assembling a range-associated matrix A and an angle-associated matrix B satisfying a bilinear model: Yₗ=AXₗB+Nₗ, l=1, . . . , L, where Xₗ∈ [symbol] is an unknown matrix of complex amplitudes associated with reflections from various range-angle pairs, M being a number of general radial distances, P being a number of general azimuth angles with respect to the radar, and Nₗ is a matrix defined by noise.

13. The method of claim 11, wherein said preprocessing further comprises utilizing prior knowledge of human typical pulse and breathing frequencies and defining a filter applied on an input set of said data matrices,
$${\{ {Y}_{l} \}}_{l = 1}^{L} ,$$
to reduce non-human clutter.

14. The method of claim 12, wherein said iterative estimation of the localization of the one or more subjects by exploiting joint sparsity and cardiopulmonary properties of said data matrix utilizes a fast iterative soft-thresholding (FISTA) algorithm to perform the following minimization:
$$𝕏 = {arg min}_{𝕏 ∈ {ℂ}^{M \times P \times L}} \frac{1}{2 L} {\sum }_{l = 1}^{L} {‖ {Y}_{l} - {AX}_{l} B ‖}_{F}^{2} + γ {‖ 𝕏 ‖}_{2 , 1}$$
where ∥ [symbol] ∥_{2,1} refers to the sum of all l₂ norms over the frame dimension of a 3D tensor [symbol] ∈ [symbol] which concatenates the frames of
$${\{ {X}_{l} \}}_{l = 1}^{L}$$
and γ≥0 is a regularization parameter, said minimization providing the unknown set
$${\{ {X}_{l} \}}_{l = 1}^{L} .$$

15. The method of claim 14, wherein said iterative estimation of the localization is accelerated by using an update step where two previous estimates are linearly combined replacing
$${X}_{l}^{( i + 1 )} by {Z}_{l}^{( i + 1 )} = {X}_{l}^{( i )} + \frac{{t}^{{( i )}_{- 1}}}{{t}^{( i + 1 )}} ( {X}_{l}^{( i )} - {X}_{l}^{( i - 1 )} )$$
where t⁽ⁱ⁺¹⁾=0.5 (1+\sqrt{1+4t⁽ⁱ⁾²}) initialized to t=0.

16. The method of claim 14, wherein said support recovery data indicative of range and angle indices of the one or more subjects is estimated by determining an average power across the frames of
$${\{ {X}_{l} \}}_{l = 1}^{L}$$
resulting in a 2D range-angle localization map.

17. The method of claim 12, wherein said processing of each said l-th data matrix Yₗ∈ [symbol] by utilizing the trained neural network with the predetermined number of layers comprises replacing the matrices A and B with weight matrices {Wᵢ} of appropriate sizes, said weight matrices being learned from train data.

18. The method of claim 17, wherein said weight matrices {Wᵢ} of appropriate sizes comprise a set
$${\{ {W}_{1}^{( i )} , {W}_{2}^{( i )} , {W}_{3}^{( i )} , {W}_{4}^{( i )} \}}_{i = 1}^{I} ,$$
I being the number of layers, wherein said weight matrices {Wᵢ} are initialized to:
$${W}_{1}^{( i )} = \frac{1}{{L}_{f}} {A}^{H} , {W}_{2}^{( i )} = {B}^{H} , {W}_{3}^{( i )} = - \frac{1}{{L}_{f}} {A}^{H} A , and {W}_{4}^{( i )} = {BB}^{H} ,$$
where L_{f} is a constant set to L_{f}=λₘₐₓ(A^{H}A)λₘₐₓ(B^{H}B) with λₘₐₓ denoting the largest singular value.

19. The method of claim 18, wherein said matrix
$${W}_{3}^{( i )}$$
is omitted and said matrix
$${W}_{4}^{( i )}$$
is reinitialized to
$${\tilde{W}}_{4}^{( i )} = - \frac{N}{{L}_{f}} {BB}^{H} .$$

20. The method of claim 19, wherein said preprocessing further comprises applying a filter on an input set of said data matrices
$${\{ {Y}_{l} \}}_{l = 1}^{L}$$
to reduce non-human clutter, said filter being learned from said training data and initialized utilizing prior knowledge on cardiopulmonary frequencies at rest.
